(19) United States
(12) Patent Application Publication
(10) Pub. No.: US 2024/0148725 A1
(43) Pub. Date: May 9, 2024

(54) COMBINATIONS FOR TREATMENT OF CANCERS RESISTANT TO EGFR-TARGETED THERAPIES

(71) Applicant: Ohio State Innovation Foundation, Columbus, OH (US)

(72) Inventor: Vidhya Karivedu, Columbus, OH (US)

(21) Appl. No.: 18/501,323

(22) Filed: Nov. 3, 2023

Related U.S. Application Data
(60) Provisional application No. 63/422,257, filed on Nov. 3, 2022.

(51) Int. Cl. A61K 31/498; A61P 35/00; C07K 16/22

(52) U.S. Cl. CPC A61K 31/498; A61P 35/00; C07K 16/22; A61K 2039/505

(57) ABSTRACT

This disclosure describes methods of treating cancers, such as head and neck cancers, resistant to an inhibitor of epidermal growth factor receptor (EGFR), such as cetuximab, by administering the EGFR inhibitor in combination or alternation with a fibroblast growth factor receptor (FGFR) inhibitor, such as erdafitinib.

### CROSS-REFERENCE TO RELATED APPLICATIONS

This application claims the benefit of priority to U.S. Provisional Application No. 63/422,257 filed Nov. 3, 2022, the disclosure of which is incorporated herein by reference in its entirety.

### TECHNICAL FIELD

This disclosure relates to methods of treating medical disorders and, more particularly, to methods of treating cancers resistant to an inhibitor of epidermal growth factor receptor (EGFR).

### BACKGROUND

Cetuximab is an anti-EGFR monoclonal antibody used for the treatment of head and neck cancer. In recurrent metastatic head and neck squamous cell carcinoma, the EXTREME regimen (cetuximab, 5-fluorouracil, and cisplatin) has been the standard of care for the past ten years. Despite the success of using cetuximab or other EGFR inhibitors in treating patients harboring specific EGFR mutations, all responding patients eventually develop acquired resistance. There is a clear need for the development of novel therapeutic protocols that can be used in treating cancers, such as head and neck cancer, that have developed resistance to EGFR inhibitors, such as cetuximab.

### SUMMARY

The present disclosure provides methods for treating cancers that have developed resistance to an epidermal growth factor receptor (EGFR) inhibitor by administering the EGFR inhibitor in combination or alternation with a fibroblast growth factor receptor (FGFR) inhibitor.

Thus, in one aspect, a method is provided of treating a cancer in a subject in need thereof,

wherein the cancer is resistant to treatment with an epidermal growth factor receptor (EGFR) inhibitor,

the method comprising administering to the subject a therapeutically effective amount of a fibroblast growth factor receptor (FGFR) inhibitor in combination or alternation with a therapeutically effective amount of the EGFR inhibitor.

In another aspect, a method is provided of treating a cancer in a subject in need thereof comprising:
a) determining if the cancer is resistant to treatment with an epidermal growth factor receptor (EGFR) inhibitor, and
b) if the cancer is determined to be resistant to treating with the EGFR inhibitor in a), administering to the subject a therapeutically effective amount of a fibroblast growth factor receptor (FGFR) inhibitor in combination or alternation with a therapeutically effective amount of the EGFR inhibitor.

In a further aspect, a method is provided of treating a cancer in a subject in need thereof,

wherein the subject has been previously administered an epidermal growth factor receptor (EGFR) inhibitor, and

wherein the cancer has developed resistance to treatment with the EGFR inhibitor,

the method comprising administering a therapeutically effective amount of a fibroblast growth factor receptor (FGFR) inhibitor in combination or alternation with a therapeutically effective amount of the EGFR inhibitor.

In some aspects, the cancer comprises head and neck cancer. In some aspects, the EGFR inhibitor comprises cetuximab. In some aspects, the FGFR inhibitor comprises erdafitinib.

The details of one or more aspects of the disclosure are set forth in the accompanying drawings and the description below. Other features, objects, and advantages of the disclosure will be apparent from the description, drawings, and claims.

### DESCRIPTION OF DRAWINGS

FIG. 1 shows the tumor volume (mm³) at treatment start (day 0) for head squamous cell carcinoma patient derived xenografts treated with control (saline+HPBCD), cetuximab, erdafitinib, and cetuximab+erdafitinib as described in the examples.

FIGS. 2A and 2B show the tumor volume in mm³ (FIG. 2A) and the percent change in tumor volume (FIG. 2B) for all cohorts as compared to the start of treatment as described in the examples.

FIG. 3 shows the percent change in body weight for all cohorts as compared to the start of treatment as described in the examples.

FIGS. 4A and 4B show the percent change in tumor volume (FIG. 4A) and the tumor volume (FIG. 4B) for the control cohort as described in the examples.

FIGS. 5A and 5B show the percent change in tumor volume (FIG. 5A) and the tumor volume (FIG. 5B) for the cetuximab cohort as described in the examples.

FIGS. 6A and 6B show the percent change in tumor volume (FIG. 6A) and the tumor volume (FIG. 6B) for the erdafitinib cohort as described in the examples.

FIGS. 7A and 7B show the percent change in tumor volume (FIG. 7A) and the tumor volume (FIG. 7B) for the cetuximab+erdafitinib cohort as described in the examples.

FIGS. 8A and 8B compare the percent change in tumor volume among individual animals in the cetuximab and cetixumab+erdafitinib cohorts (FIG. 8A) and in the erdafitinib and cetuximab+erdafitinib cohorts (FIG. 8B) as described in the examples.

FIG. 9 shows the tumor weight at the end of the study for each treatment cohort as described in the examples.

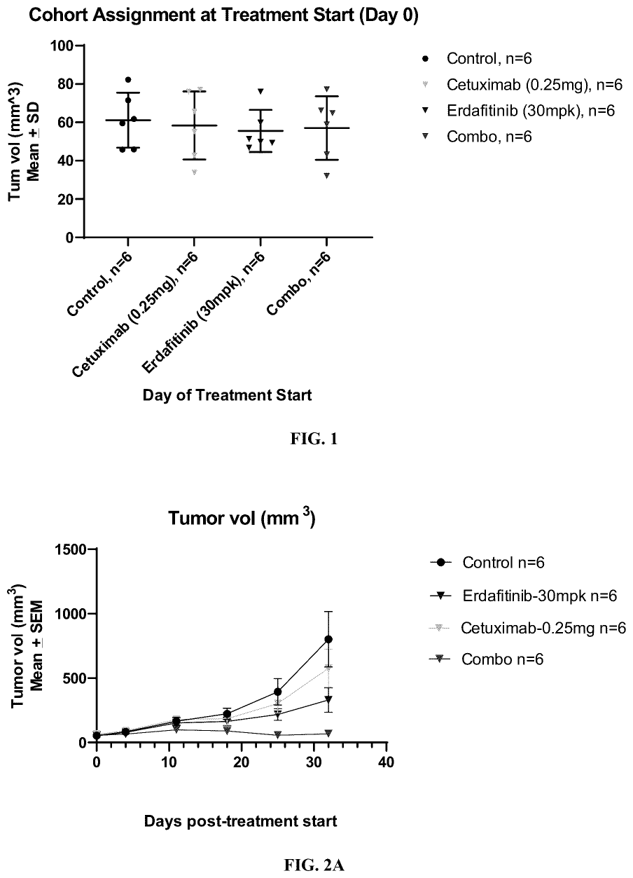

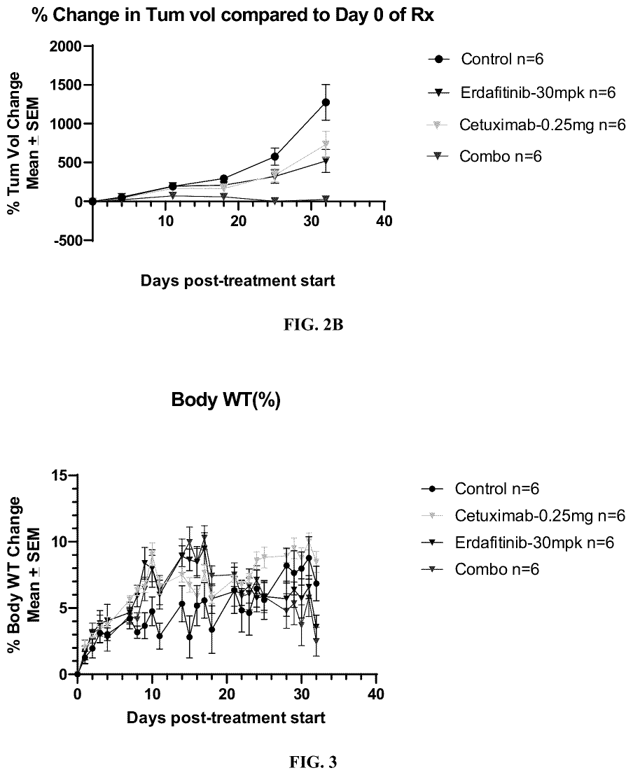

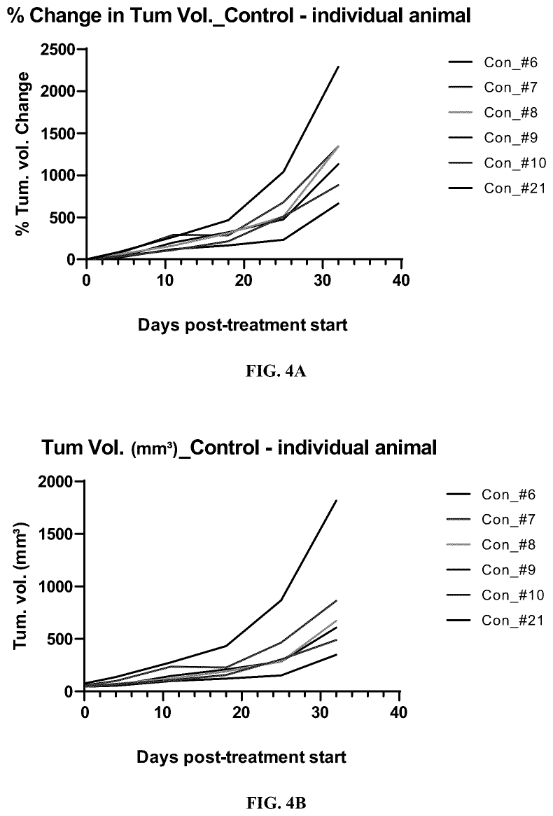

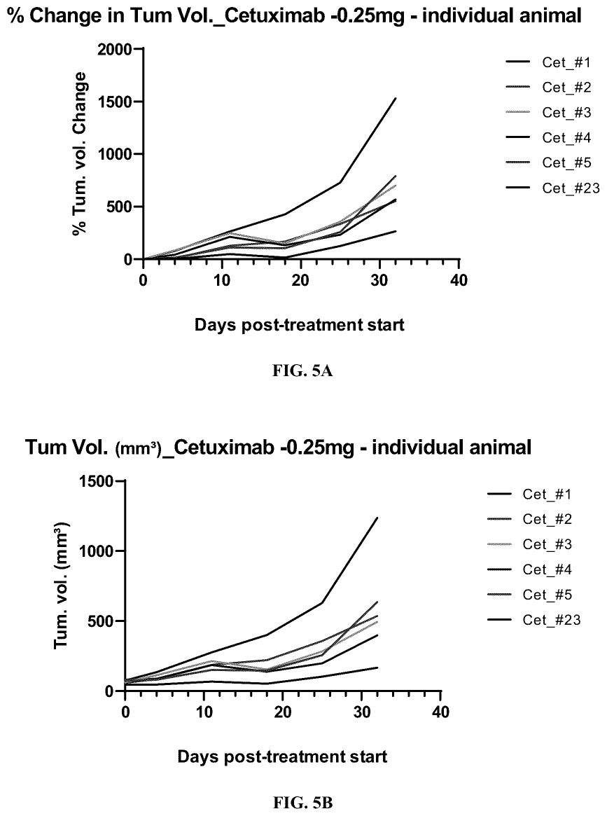

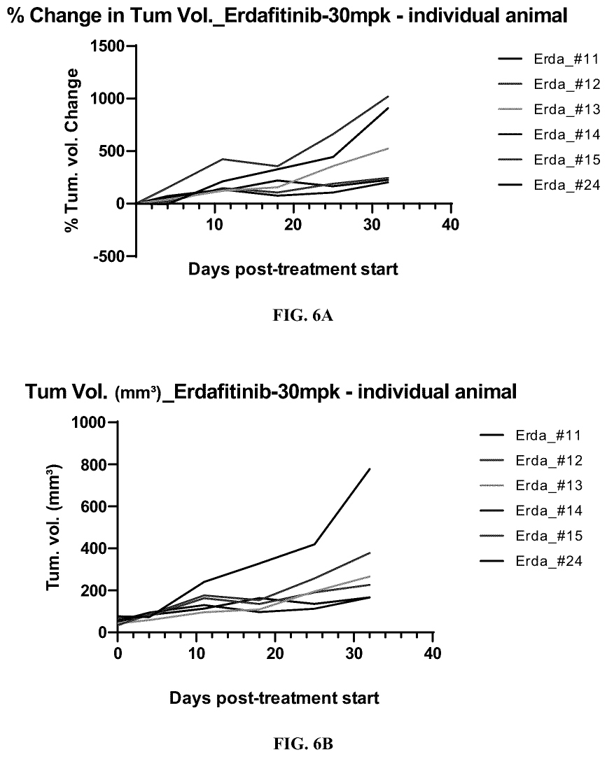

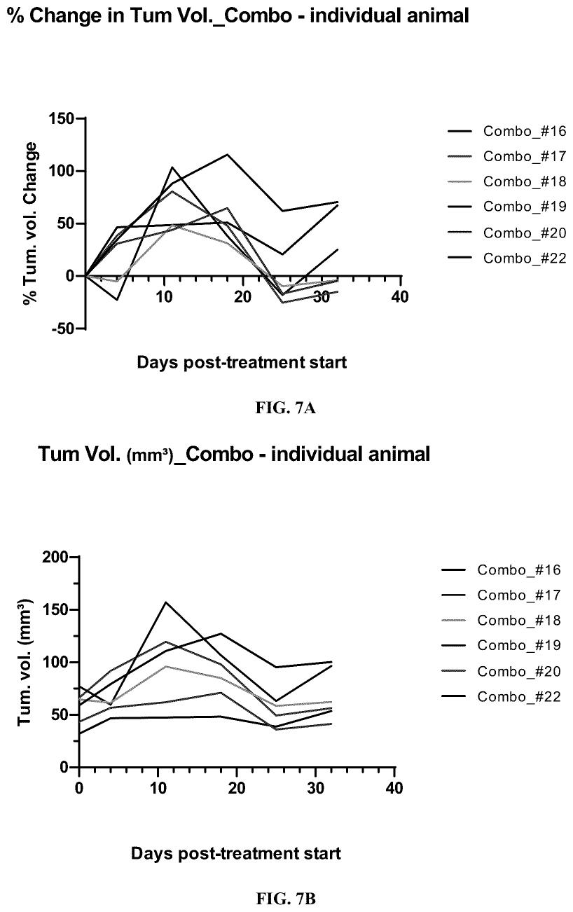

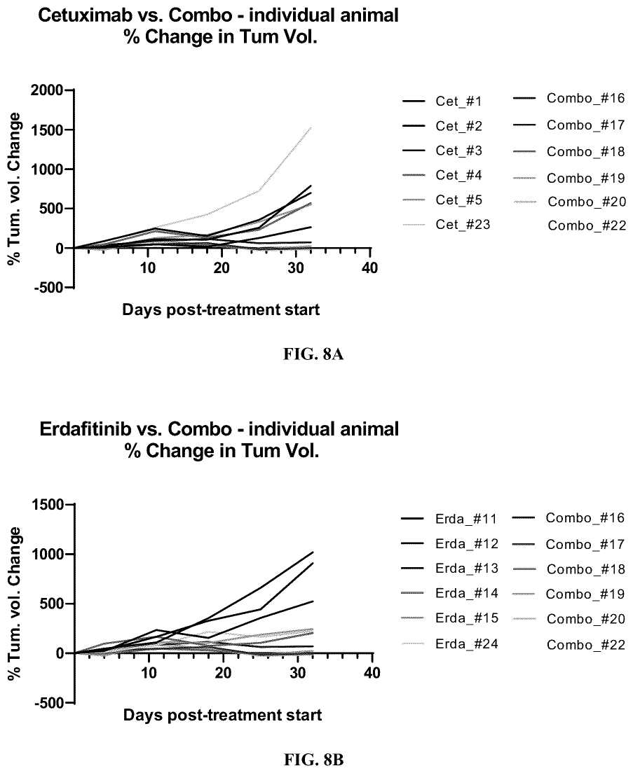

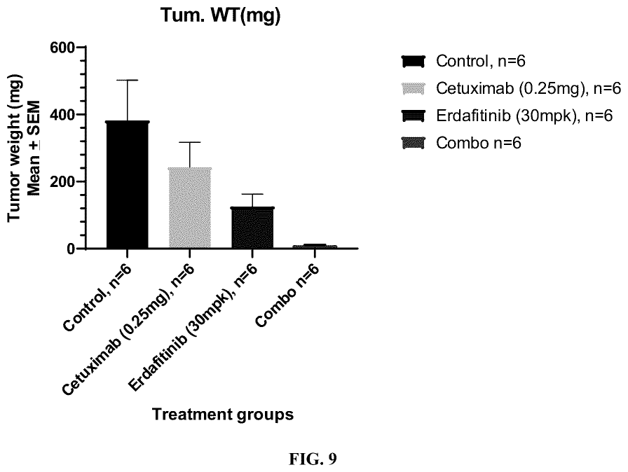

Like reference symbols in the various drawings indicate like elements.

### DETAILED DESCRIPTION

The following description of the disclosure is provided as an enabling teaching of the disclosure in its best, currently known aspects. Many modifications and other aspects disclosed herein will come to mind to one skilled in the art to which the disclosed compositions and methods pertain, having the benefit of the teachings presented in the foregoing descriptions and the associated drawings. Therefore, it is to be understood that the disclosures are not to be limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual aspects described and illustrated herein has discrete components and features that may be readily separated from or combined with the features of any of the other several aspects without departing from the scope or spirit of the present disclosure.

Any recited method can be carried out in the order of events recited or in any other order that is logically possible. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to but does not preclude the presence or addition of one or more features, integers, steps, or components, or groups thereof. Moreover, each of the terms “by,” “comprising,” “comprises,” “comprised of” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of” Similarly, the term “consisting essentially of” is intended to include examples encompassed by the term “consisting of”.

As used in the specification and the appended claims, the singular forms “a,” “an,” and “the” include plural referents unless the context clearly dictates otherwise. Thus, for example, reference to “an inhibitor,” “a composition,” or “a cancer” includes, but is not limited to, two or more such inhibitors, compositions, or cancers, and the like.

It should be noted that ratios, concentrations, amounts, and other numerical data can be expressed herein in a range format. It can be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. Ranges can be expressed herein as from “about” one particular value and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x,“about y’, and ‘about z’ as well as the ranges of ‘less than x,’ less than y′, and ‘less than z.’ Likewise, the phrase ‘about x, y, z, or greater’ should be interpreted to include the specific ranges of ‘about x,”about y’, and ‘about z’ as well as the ranges of ‘greater than x,’ greater than y′, and ‘greater than z.’ In addition, the phrase “about ‘x’ to ‘y’,” where ‘x’ and ‘y’ are numerical values, includes “about ‘x’ to about ‘y’.”

It is to be understood that such a range format is used for convenience and brevity and, thus, should be interpreted in a flexible manner to include not only the numerical values explicitly recited as the limits of the range but also all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5% but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter, or other quantity or characteristic is “about,” “approximate,” or “at or about,” whether or not expressly stated to be such. It is understood that where “about,” “approximate,” or “at or about” is used before a quantitative value, the parameter also includes the specific quantitative value itself unless specifically stated otherwise.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors, including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single-dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art, however, that a patient may insist upon a lower dose or tolerable dose for medical reasons, psychological reasons, or virtually any other reasons.

A response to a therapeutically effective dose of a disclosed compound or composition can be measured by determining the physiological effects of the treatment or medication, such as the decrease or lack of disease symptoms following the administration of the treatment or pharmacological agent. Other assays will be known to one of ordinary skill in the art and can be employed for measuring the level of the response. The amount of a treatment may be varied, for example, by increasing or decreasing the amount of a disclosed compound and/or pharmaceutical composition, by changing the disclosed compound and/or pharmaceutical composition administered, changing the route of administration, changing the dosage timing, and so on. Dosage can vary and can be administered in one or more dose administrations daily for one or several days. Guidance can be found in the literature for appropriate dosages for given classes of pharmaceutical products.

As used herein, the terms “optional” or “optionally” mean that the subsequently described event or circumstance can or cannot occur and that the description includes instances where said event or circumstance occurs and instances where it does not.

As used interchangeably herein, “subject,” “individual,” or “patient” can refer to a vertebrate organism, such as a mammal (e.g., human). “Subject” can also refer to a cell, a population of cells, a tissue, an organ, or an organism, preferably to a human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom, or condition thereof, such as a cancer. The effect can be therapeutic in terms of a partial or complete cure of a disease, condition, symptom, or adverse effect attributed to the disease, disorder, or condition. The term “treatment” as used herein can include any treatment of a disorder in a subject, particularly a human, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment,” as used herein, can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating” can include inhibiting the disease, disorder, or condition, e.g., impeding its progress, and relieving the disease, disorder, or condition, e.g., causing regression of the disease, disorder, and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect or to decreasing the rate of advancement of a disease, disorder, condition, or side effect.

The present disclosure provides methods for treating cancers resistant to treatment with an epidermal growth factor receptor (EGFR) inhibitor by administering the EGFR inhibitor in combination or alternation with a fibroblast growth factor receptor (FGFR) inhibitor.

Thus, in one aspect, a method is provided of treating a cancer in a subject in need thereof,

wherein the cancer is resistant to treatment with an epidermal growth factor receptor (EGFR) inhibitor,

the method comprising administering to the subject a therapeutically effective amount of a fibroblast growth factor receptor (FGFR) inhibitor in combination or alternation with a therapeutically effective amount of the EGFR inhibitor.

In another aspect, a method is provided of treating a cancer in a subject in need thereof comprising:
a) determining if the cancer is resistant to treatment with an epidermal growth factor receptor (EGFR) inhibitor, and
b) if the cancer is determined to be resistant to treating with the EGFR inhibitor in a), administering to the subject a therapeutically effective amount of a fibroblast growth factor receptor (FGFR) inhibitor in combination or alternation with a therapeutically effective amount of the EGFR inhibitor.

In another aspect, a method is provided of treating a cancer in a subject in need thereof,

wherein the subject has been previously administered an epidermal growth factor receptor (EGFR) inhibitor, and wherein the cancer has developed resistance to treatment with the EGFR inhibitor, the method comprising administering a therapeutically effective amount of a fibroblast growth factor receptor (FGFR) inhibitor in combination or alternation with a therapeutically effective amount of the EGFR inhibitor.

In some aspects, the EGFR inhibitor is selected from cetuximab, panitumumab, zalutumumab, nimotuzumab, matuzumab, gefitinib, erlotinib, brigatinib, lapatinib, and osimertinib. In one aspect, the EGFR inhibitor is cetuximab. In one aspect, the EGFR inhibitor is panitumumab. In one aspect, the EGFR inhibitor is zalutumumab. In one aspect, the EGFR inhibitor is nimotuzumab. In one aspect, the EGFR inhibitor is matuzumab. In one aspect, the EGFR inhibitor is gefitinib. In one aspect, the EGFR inhibitor is erlotinib. In one aspect, the EGFR inhibitor is brigatinib. In one aspect, the EGFR inhibitor is lapatinib. In one aspect, the EGFR inhibitor is osimertinib.

In some aspects, the FGFR inhibitor is selected from pemigatinib, erdafitinib, infigratinib, derazantinib, and futibatinib. In one aspect, the FGFR inhibitor is pemigatinib. In one aspect, the FGFR inhibitor is erdafitinib. In one aspect, the FGFR inhibitor is infigratinib. In one aspect, the FGFR inhibitor is derazantinib. In one aspect, the FGFR inhibitor is futibatinib.

In another aspect, a method is provided of treating a cancer in a subject in need thereof,

wherein the cancer is resistant to treatment with cetuximab, the method comprising administering to the subject a therapeutically effective amount of erdafitinib in combination or alternation with a therapeutically effective amount of cetuximab.

In another aspect, a method is provided of treating a cancer in a subject in need thereof comprising:
a) determining if the cancer is resistant to treatment with cetuximab, and
b) if the cancer is determined to be resistant to treating with cetuximab in a), administering to the subject a therapeutically effective amount of erdafitinib in combination or alternation with a therapeutically effective amount of cetuximab.

In another aspect, a method is provided of treating a cancer in a subject in need thereof,

wherein the subject has been previously cetuximab, and

wherein the cancer has developed resistance to treatment with cetuximab,

the method comprising administering a therapeutically effective amount of erdafitinib in combination or alternation with a therapeutically effective amount of cetuximab.

The term “neoplasia” or “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue, and most invade surrounding tissues, can metastasize to several sites, are likely to recur after attempted removal, and may cause the death of the patient unless adequately treated. As used herein, the term neoplasia is used to describe all cancerous disease states and embraces or encompasses the pathological process associated with malignant, hematogenous, ascitic, and solid tumors. The cancers that may be treated by the compositions disclosed herein may comprise carcinomas, sarcomas, lymphomas, leukemias, germ cell tumors, or blastomas.

Carcinomas which may be treated by the compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, and carcinoma vilosum.

Representative sarcomas which may be treated by the compositions of the present disclosure include but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma(MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compositions of the present disclosure may be used in the treatment of a lymphoma. Lymphomas that may be treated include mature B cell neoplasms, mature T cell and natural killer (NK) cell neoplasms, precursor lymphoid neoplasms, Hodgkin lymphomas, and immunodeficiency-associated lymphoproliferative disorders.

Representative mature B cell neoplasms include, but are not limited to, B-cell chronic lymphocytic leukemia/small cell lymphoma, B-cell prolymphocytic leukemia, lymphoplasmacytic lymphoma (such as Waldenstrom macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lyphomatoid granulomatosis, primary mediastinal (thymic) large B-cell lymphoma, intravascular large B-cell lymphoma, ALK+large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compositions of the present disclosure may be used in the treatment of a Leukemia. Representative examples of leukemias include but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compositions of the present disclosure may be used in the treatment of a germ cell tumor, for example, germinomatous (such as germinoma, dysgerminoma, and seminoma), non-germinomatous (such as embryonal carcinoma, endodermal sinus tumor, choriocarcinoma, teratoma, polyembryoma, and gonadoblastoma) and mixed tumors.

The compositions of the present disclosure may be used in the treatment of blastomas, for example, hepatoblastoma, medulloblastoma, nephroblastoma, neuroblastoma, pancreatoblastoma, pleuropulmonary blastoma, retinoblastoma, and glioblastoma multiforme.

Representative cancers which may be treated include, but are not limited to: bone and muscle sarcomas such as chondrosarcoma, Ewing's sarcoma, malignant fibrous histiocytoma of bone/osteosarcoma, osteosarcoma, rhabdomyosarcoma, and heart cancer; brain and nervous system cancers such as astrocytoma, brainstem glioma, pilocytic astrocytoma, ependymoma, primitive neuroectodermal tumor, cerebellar astrocytoma, cerebral astrocytoma, glioma, medulloblastoma, neuroblastoma, oligodendroglioma, pineal astrocytoma, pituitary adenoma, and visual pathway and hypothalamic glioma; breast cancers including invasive lobular carcinoma, tubular carcinoma, invasive cribriform carcinoma, medullary carcinoma, male breast cancer, Phyllodes tumor, and inflammatory breast cancer; endocrine system cancers such as adrenocortical carcinoma, islet cell carcinoma, multiple endocrine neoplasia syndrome, parathyroid cancer, phemochromocytoma, thyroid cancer, and Merkel cell carcinoma; eye cancers including uveal melanoma and retinoblastoma; gastrointestinal cancers such as anal cancer, appendix cancer, cholangiocarcinoma, gastrointestinal carcinoid tumors, colon cancer, extrahepatic bile duct cancer, gallbladder cancer, gastric cancer, gastrointestinal stromal tumor, hepatocellular cancer, pancreatic cancer, and rectal cancer; genitourinary and gynecologic cancers such as bladder cancer, cervical cancer, endometrial cancer, extragonadal germ cell tumor, ovarian cancer, ovarian epithelial cancer, ovarian germ cell tumor, penile cancer, renal cell carcinoma, renal pelvis and ureter transitional cell cancer, prostate cancer, testicular cancer, gestational trophoblastic tumor, urethral cancer, uterine sarcoma, vaginal cancer, vulvar cancer, and Wilms tumor; head and neck cancers such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer; hematopoietic cancers such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, hepatosplenic T-cell lymphoma, Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, Mast cell leukemia, mediastinal large B cell lymphoma, multiple myeloma/plasma cell neoplasm, myelodysplastic syndroms, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sezary syndrome, splenic marginal zone lymphoma, and T-cell prolymphocytic leukemia; skin cancers such as basal cell carcinoma, squamous cell carcinoma, skin adnexal tumors (such as sebaceous carcinoma), melanoma, Merkel cell carcinoma, sarcomas of primary cutaneous origin (such as dermatofibrosarcoma protuberans), and lymphomas of primary cutaneous origin (such as mycosis fungoides); thoracic and respiratory cancers such as bronchial adenomas/carcinoids, small cell lung cancer, mesothelioma, non-small cell lung cancer, pleuropulmonary blastoma, laryngeal cancer, and thymoma or thymic carcinoma; HIV/AIDs-related cancers such as Kaposi sarcoma; epithelioid hemangioendothelioma; desmoplastic small round cell tumor; and liposarcoma.

In particular aspects, the cancer comprises head and neck cancer (e.g., head and neck squamous cell carcinoma). In particular aspects, the cancer comprises recurrent metastatic head and neck squamous cell carcinoma.

In another aspect, a method is provided of treating head and neck cancer in a subject in need thereof,

wherein the head and neck cancer is resistant to treatment with cetuximab,

the method comprising administering to the subject a therapeutically effective amount of erdafitinib in combination or alternation with a therapeutically effective amount of cetuximab.

In another aspect, a method is provided of treating head and neck cancer in a subject in need thereof comprising:
a) determining if the head and neck cancer is resistant to treatment with cetuximab, and
b) if the head and neck cancer is determined to be resistant to treating with cetuximab in a), administering to the subject a therapeutically effective amount of erdafitinib in combination or alternation with a therapeutically effective amount of cetuximab.

In another aspect, a method is provided of treating head and neck cancer in a subject in need thereof,

wherein the subject has been previously cetuximab, and

wherein the head and neck cancer has developed resistance to treatment with cetuximab,

the method comprising administering a therapeutically effective amount of erdafitinib in combination or alternation with a therapeutically effective amount of cetuximab.

The methods can further comprise administering one or more additional therapeutic agents, for example, anticancer agents or anti-inflammatory agents. Additionally, the method can further comprise administering a therapeutically effective amount of ionizing radiation to the subject.

Methods of killing a cancer or tumor cell, wherein the cancer or tumor cell is resistant to killing with administration of an EGFR inhibitor alone, are also provided comprising contacting the cancer or tumor cell with an effective amount of EGFR inhibitor in combination or alternation with an effective amount of an FGFR inhibitor. The methods can further include administering one or more additional therapeutic agents or administering an effective amount of ionizing radiation.

The disclosed methods can optionally include identifying a patient who is or can be in need of treatment of an oncological disorder. The patient can be a human or other mammal, such as a primate (monkey, chimpanzee, ape, etc.), dog, cat, cow, pig, or horse, or other animals having an oncological disorder. In some aspects, the subject can receive the therapeutic compositions prior to, during, or after surgical intervention to remove part or all of a tumor.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can also be systemically administered, such as intravenously or orally, optionally in combination with a pharmaceutically acceptable carrier, such as an inert diluent, or an assimilable edible carrier for oral delivery. In addition, the active compound can be incorporated into sustained-release preparations and/or devices.

For the treatment of oncological disorder, compounds, agents, and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds, agents, and compositions disclosed herein can be used in methods of treating cancer wherein the patient is to be treated or is or has been treated with mitotic inhibitors such as taxol or vinblastine, alkylating agents such as cyclophosphamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anticancer drugs or antibodies, such as, for example, imatinid or trastuzumab. These other substances or radiation treatments can be given at the same time as or at different times from the compounds disclosed herein. Examples of other suitable chemotherapeutic agents include but are not limited to, altretamine, bleomycin, bortezomib, busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxyurea, idarubicin, ifosfamide, imatinib, irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uraxil, temozolomide, thiotepa, tioguanine/thioguanine, topotexan, treosulfan, vinblastine, vincristine, vindesine, and vinorelbine. Examples of suitable immunotherapeutic agents include, but are not limited to, alemtuzumab, gemtuzumab, iodine 131 tositumomab, rituximab, and trastuzumab. Cytotoxic agents include, for example, radioactive isotopes and toxins of bacterial, fungal, plant, or animal origin. Also disclosed are methods of treating an oncological disorder comprising administering an effective amount of a compound described herein prior to, subsequent to, and/or in combination with administration of a chemotherapeutic agent, an immunotherapeutic agent, a radiotherapeutic agent, or radiotherapy.

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art, including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration or at continuous and distinct intervals as can be readily determined by a person skilled in the art.

Compositions, as described herein, comprising an active compound and a pharmaceutically acceptable carrier or excipient of some sort, may be useful in a variety of medical and non-medical applications. For example, pharmaceutical compositions comprising an active compound and an excipient may be useful for the treatment or prevention of a cancer in a subject in need thereof.

“Pharmaceutically acceptable carrier” (sometimes referred to as a “carrier”) means a carrier or excipient that is useful in preparing a pharmaceutical or therapeutic composition that is generally safe and nontoxic and includes a carrier that is acceptable for veterinary and/or human pharmaceutical or therapeutic use. The terms “carrier” or “pharmaceutically acceptable carrier” can include, but are not limited to, phosphate-buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well-known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants, and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any nontoxic, inert solid, semisolid, or liquid filler, diluent, encapsulating material, or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other nontoxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, the time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some aspects, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water-insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxy ethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha-tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain aspects, the preservative is an antioxidant. In other aspects, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof. Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cottonseed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, varoius gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy (Polyethyleneglycol)-2000], and 1,2-Distearoyl-sn-glycero-3-Phosphoethanolamine-N-[Methoxy(Polyethylene glycol)-5000]), copolymers and salts thereof. Additionally, the composition may further comprise an emulsifying agent.

Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain aspects, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions, may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P., and isotonic sodium chloride solution. In addition, sterile, fixed oils are conventionally employed as a solvent or suspending medium. Any bland fixed oil can be employed, including synthetic mono- or diglycerides. In addition, fatty acids such as oleic acid are used in the preparation of injectables. In certain aspects, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter or by incorporating sterilizing agents in the form of sterile solid compositions, which can be dissolved or dispersed in sterile water or other sterile injectable media prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate-controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors, including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some aspects, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors, including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity and in vivo activity in animal models. Methods for the extrapolation of effective dosages in mice and other animals to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex, and extent of the disease in the patient and can be determined by one skilled in the art. The dosage can be adjusted by the individual physician in the event of any counterindications. Dosage can vary and can be administered in one or more dose administrations daily for one or several days.

Kits for practicing the methods described herein are further provided. By “kit” is intended any manufacture (e.g., a package or a container) comprising at least one reagent, e.g., any one of the compounds described herein. The kit can be promoted, distributed, or sold as a unit for performing the methods described herein. Additionally, the kits can contain a package insert describing the kit and methods for its use. Any or all of the kit reagents can be provided within containers that protect them from the external environment, such as in sealed containers or pouches.

To provide for the administration of such dosages for the desired therapeutic treatment, in some aspects, pharmaceutical compositions disclosed herein can comprise between 0.1% and 45%, and especially 1 and 15%, by weight of the total of one or more of the compounds based on the weight of the total composition including carriers and/or diluents. Illustratively, dosage levels of the administered active ingredients can be intravenous 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasally, 0.01 to about 20 mg/kg; and aerosol, 0.01 to about 20 mg/kg of animal (body) weight.

Also disclosed are kits that comprise a composition comprising a compound disclosed herein in one or more containers. The disclosed kits can optionally include pharmaceutically acceptable carriers and/or diluents. In one aspect, a kit includes one or more other components, adjuncts, or adjuvants, as described herein. In another aspect, a kit includes one or more anticancer agents, such as those agents described herein. In one aspect, a kit includes instructions or packaging materials that describe how to administer a compound or composition of the kit. Containers of the kit can be of any suitable material, e.g., glass, plastic, metal, etc., and of any suitable size, shape, or configuration. In one aspect, a compound and/or agent disclosed herein is provided in the kit as a solid, such as a tablet, pill, or powder form. In another aspect, a compound and/or agent disclosed herein is provided in the kit as a liquid or solution. In one aspect, the kit comprises an ampoule or syringe containing a compound and/or agent disclosed herein in liquid or solution form.

In view of the described methods and compositions, hereinbelow are described certain more particularly described aspects of the disclosures. These particularly recited aspects should not, however, be interpreted to have any limiting effect on any different claims containing different or more general teachings described herein or that the “particular” aspects are somehow limited in some way other than the inherent meanings of the language literally used therein.
Aspect 1. A method of treating a cancer in a subject in need thereof, wherein the cancer is resistant to treatment with an epidermal growth factor receptor (EGFR) inhibitor, the method comprising administering to the subject a therapeutically effective amount of a fibroblast growth factor receptor (FGFR) inhibitor in combination or alternation with a therapeutically effective amount of the EGFR inhibitor.
Aspect 2. A method of treating a cancer in a subject in need thereof comprising:
   a) determining if the cancer is resistant to treatment with an epidermal growth factor receptor (EGFR) inhibitor, and
   b) if the cancer is determined to be resistant to treating with the EGFR inhibitor in a), administering to the subject a therapeutically effective amount of a fibroblast growth factor receptor (FGFR) inhibitor in combination or alternation with a therapeutically effective amount of the EGFR inhibitor.
Aspect 3. A method of treating a cancer in a subject in need thereof, wherein the subject has been previously administered an epidermal growth factor receptor (EGFR) inhibitor, and wherein the cancer has developed resistance to treatment with the EGFR inhibitor, the method comprising administering a therapeutically effective amount of a fibroblast growth factor receptor (FGFR) inhibitor in combination or alternation with a therapeutically effective amount of the EGFR inhibitor.
Aspect 4. The method of any one of aspects 1-3, wherein the subject is a human.
Aspect 5. The method of any one of aspects 1-4, wherein the EGFR inhibitor is selected from cetuximab, panitumumab, zalutumumab, nimotuzumab, matuzumab, gefitinib, erlotinib, brigatinib, lapatinib, and osimertinib.
Aspect 6. The method of any one of aspects 1-5, wherein the EGFR inhibitor is cetuximab.
Aspect 7. The method of any one of aspects 1-6, wherein the FGFR inhibitor is selected from pemigatinib, erdafitinib, infigratinib, derazantinib, and futibatinib.
Aspect 8. The method of any one of aspects 1-7, wherein the FGFR inhibitor is erdafitinib.
Aspect 9. The method of any one of aspects 1-8, wherein the cancer is head and neck cancer.
Aspect 10. The method of any one of aspects 1-9, wherein the cancer is recurrent metastatic head and neck squamous cell carcinoma.
Aspect 11. The method of any one of aspects 1-10, further comprising administering one or more additional therapeutic agents, such as additional anticancer agents or anti-inflammatory agents.
Aspect 12. The method of any one of aspects 1-11, further comprising administering a therapeutically effective amount of ionizing radiation to the subject.

A number of aspects of the disclosure have been described. Nevertheless, it will be understood that various modifications may be made without departing from the spirit and scope of the invention. Accordingly, other aspects are within the scope of the following claims.

By way of non-limiting illustration, examples of certain aspects of the present disclosure are given below.

### Examples

The following examples are put forth to provide those of ordinary skill in the art with a complete disclosure and description of how the methods claimed herein are made and evaluated and are intended to be purely exemplary of the invention and are not intended to limit the scope of what the inventors regard as their invention. Efforts have been made to ensure accuracy concerning numbers (e.g., amounts, temperature, etc.), but some errors and deviations should be accounted for.

### Evaluation of Erdafitinib and Cetuximab Combination in a Head Squamous Cell Carcinoma Patient-Derived Xenograft Model

The effect of the combination of erdafitinib and cetuximab in the treatment of head squamous cell carcinoma patient-derived xenografts (TM01145 from Jackson Laboratory, subcutaneous injection) was evaluated in NSG mice. The mice were randomly assigned to the following cohorts (n=6 for each cohort):

control cohort administered pharmaceutical-grade saline (500 uL per mouse) intraperitoneally twice a week on Monday and Friday and 10% 2-hydroxypropyl-beta-cyclodextrin (125 uL per mouse) orally five days a week on Monday to Friday;

cetuximab (0.5 mg/mL in pharmaceutical-grade saline, 0.25 mg in 500 uL vol per mouse administered intraperitoneally twice a week on Monday and Friday) administered intraperitoneally twice a week on Monday and Friday;

erdafitinib (60 mg/mL in 10% hydroxypropyl-beta-cyclodextrin, 30 mg per kg) administered orally five days a week on Monday to Friday; and

cetuximab (0.5 mg/mL in pharmaceutical-grade saline, 0.25 mg in 500 uL vol per mouse administered intraperitoneally twice a week on Monday and Friday) administered intraperitoneally twice a week on Monday and Friday and erdafitinib (60 mg/mL in 10% hydroxypropyl-beta-cyclodextrin, 30 mg per kg) administered orally five days a week on Monday to Friday. Treatment was started when tumors became palpable and reached approximately 60 mm³ in size on average for each cohort. Tumors were measured once a week with digital calipers, and tumor volume was calculator using the formula (w²×1)/2, where “1” represents the max diameter of the tumor and “w” represents the width of the tumor perpendicular to “1”. The percent change in tumor volume was calculated using the formula (tumor vol at a given day post treatment start/tumor volume on first day of treatment)×100. The percent change in body weight of all animals during treatment was measured. All cohorts were euthanized at 32 days after treatment start, tumors were harvested, and then weighed.

The compositions and methods of the appended claims are not limited in scope by the specific compositions and methods described herein, which are intended as illustrations of a few aspects of the claims, and any compositions and methods that are functionally equivalent are intended to fall within the scope of the claims. Various modifications of the compositions and methods, in addition to those shown and described herein, are intended to fall within the scope of the appended claims. Further, while only certain representative compositions and method steps disclosed herein are specifically described, other combinations of the compositions and method steps are also intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

What is claimed is:

1. A method of treating a cancer in a subject in need thereof,
wherein the cancer is resistant to treatment with an epidermal growth factor receptor (EGFR) inhibitor,
the method comprising administering to the subject a therapeutically effective amount of a fibroblast growth factor receptor (FGFR) inhibitor in combination or alternation with a therapeutically effective amount of the EGFR inhibitor.

2. The method of claim 1, wherein the subject is a human.

3. The method of claim 1, wherein the EGFR inhibitor is selected from cetuximab, panitumumab, zalutumumab, nimotuzumab, matuzumab, gefitinib, erlotinib, brigatinib, lapatinib, and osimertinib.

4. The method of claim 1, wherein the EGFR inhibitor is cetuximab.

5. The method of claim 1, wherein the FGFR inhibitor is selected from pemigatinib, erdafitinib, infigratinib, derazantinib, and futibatinib.

6. The method of claim 1, wherein the FGFR inhibitor is erdafitinib.

7. The method of claim 1, wherein the cancer is head and neck cancer.

8. The method of claim 1, wherein the cancer is recurrent metastatic head and neck squamous cell carcinoma.

9. A method of treating a cancer in a subject in need thereof comprising:
a) determining if the cancer is resistant to treatment with an epidermal growth factor receptor (EGFR) inhibitor, and
b) if the cancer is determined to be resistant to treating with the EGFR inhibitor in a), administering to the subject a therapeutically effective amount of a fibroblast growth factor receptor (FGFR) inhibitor in combination or alternation with a therapeutically effective amount of the EGFR inhibitor.

10. The method of claim 9, wherein the subject is a human.

11. The method of claim 9, wherein the EGFR inhibitor is cetuximab.

12. The method of claim 9, wherein the FGFR inhibitor is erdafitinib.

13. A method of treating a cancer in a subject in need thereof,
wherein the subject has been previously administered an epidermal growth factor receptor (EGFR) inhibitor, and
wherein the cancer has developed resistance to treatment with the EGFR inhibitor, the method comprising administering a therapeutically effective amount of a fibroblast growth factor receptor (FGFR) inhibitor in combination or alternation with a therapeutically effective amount of the EGFR inhibitor.

14. The method of claim 13, wherein the subject is a human.

15. The method of claim 13, wherein the EGFR inhibitor is selected from cetuximab, panitumumab, zalutumumab, nimotuzumab, matuzumab, gefitinib, erlotinib, brigatinib, lapatinib, and osimertinib.

16. The method of claim 13, wherein the EGFR inhibitor is cetuximab.

17. The method of claim 13, wherein the FGFR inhibitor is selected from pemigatinib, erdafitinib, infigratinib, derazantinib, and futibatinib.

18. The method of claim 13, wherein the FGFR inhibitor is erdafitinib.

19. The method of claim 13, wherein the cancer is head and neck cancer.

20. The method of claim 13, wherein the cancer is recurrent metastatic head and neck squamous cell carcinoma.
